(19) United States
(12) Patent Application Publication
(10) Pub. No.: US 2009/0192427 A1
(43) Pub. Date: Jul. 30, 2009

(54) WALKING SPLINT WITH ANTI-SKID BOTTOM

(75) Inventors: IVAN E. BROWN, Spirit Lake, IA (US); TERYLE L. KOUNKEL, Spirit Lake, IA (US)

Correspondence Address: MCKEE, VOORHEES & SEASE, P.L.C., 801 GRAND AVENUE, SUITE 3200, DES MOINES, IA, 50309-2721, US

(73) Assignee: BROWN MEDICAL INDUSTRIES, Spirit Lake, IA (US)

(21) Appl. No.: 12/021,642

(22) Filed: Jan. 29, 2008

(51) Int. Cl. A61F 5/00

(52) U.S. Cl. 602/27; 602/5; 602/23

(57) ABSTRACT

A walking splint removably attachable to the lower leg and foot to substantially immobilize the ankle joint and provide stretching of the plantar fascia having the improved advantage that posterior shell of the walking splint has an asymmetrically positioned anti-skid tread on the exterior of the posterior shell, preferably under both the foot area and the heel area.

### FIELD OF THE INVENTION

The present invention relates to a walking splint to mobilize the patient while the ankle joint is immobile and stretching the Plantar Fascia.

### BACKGROUND OF THE INVENTION

There are many reasons for orthopedic treatment that necessitate immobilization of the ankle joint and holding it to a fairly immobile position. One of the most common reasons is the common ankle sprain. Usually the treatment objective is to relieve the pain and immobilize the joint and the ligaments to promote healing of torn fibers.

Another common reason for needed immobilization is plantar fasciitis. Plantar fasciitis is one of the most common causes of heel pain. This condition occurs in a wide variety of individuals. Plantar fasciitis can and does develop at any age, particularly in young athletes who put extreme exertion on their feet such as basketball and volleyball players. Many patients with plantar fasciitis also have moderate pronation; about 15% have high-arched, ridge foot; and the remainder have an anatomically normal or non-affected foot. Some of the patients who undergo radiography for suspected plantar fasciitis are found to have a subcalcaneal or “bone” spur.

Evidence of the need for effective therapy is apparent when it is considered that over 95% of all heel pain is diagnosed as plantar fasciitis. Plantar fasciitis is best described as an inflammation of the ligament that runs from the heel to the ball of the foot, which helps support the arch. Patients with plantar fasciitis will experience pain, upon standing, on the bottom or inside of their heel. Typically, the pain is worse in the morning when getting out of bed and after resting when the person stands up, putting stretching pressure on the tendons.

Typically the primary anatomic cause of plantar fasciitis is some degree of microtrauma and tearing at the site of the Plantar Fascia insertion. These abnormalities, which may also be present at the origin of the Plantar Fascia, result from repetitive trauma and collagen degeneration and angiofibroblastic hyperplasia. Upon physical examination the range of motion of the affected ankle is less than that of the contralateral ankle. By pressing the thumb against the middle of the affected heel, the physician can delineate the area of the Plantar Fascial pain. Pressure similarly applied underneath the calcaneus reveals the area of subcalcaneal pain. Conservative treatment, including night splints, results in relief of plantar fasciitis in a majority of patients.

One medical method known in the art in reducing Plantar Fascial pain is to stretch the Plantar Fascia for a period of time. By keeping the Plantar Fascia on stretch, it is believed that an ultimate reduction of the internal tension of the Plantar Fascia can be achieved. Through this treatment, it is believed that the pain associated with this medical condition can be reduced, and possibly eliminated.

A typical treatment program would have the patient wear the splint while sleeping, and remove the splint immediately upon awakening in the morning. The patient will continue wearing the night splint for a 3-month period. After that time the patient will be weaned off of the splint in 2-week increments, using the device every other night, then every third night, then every fourth night, and from then on as needed.

A number of plantar fasciitis night splints are known in the art. One successful unit is commonly assigned U.S. Pat. No. 6,267,742. There are many others that have been patented. However, none have the suspension architecture or offer the comfort, ease of use, compactability, or degrees of rotation and angulation of U.S. Pat. No. 6,267,742.

By way of illustration of the splint state of the art, U.S. Pat. No. 4,649,939, issued to R. Curtis on Mar. 17, 1987 utilized over a shoe. U.S. Pat. No. 5,038,762, issued to H. Hess, et. al., on Aug. 13, 1991, teaches of a U-Shaped yoke which can be wrapped about the heel and ankle. U.S. Pat. No. 5,090,404, issued to C. Kallassy on Feb. 25, 1992, teach of another way to place a strap about the heel and foot. U.S. Pat. No. 5,257,969 issued to C. Mance on Nov. 2, 1993 teaches of a foot support which consists of a toe pouch and straps the wrap about the ankle. U.S. Pat. No. 5,425,701 issued to C. Oster et al. on Jun. 20, 1995, teaches of a boot with upright struts which attach to a foot pad designable for each patient's foot shape. U.S. Pat. No. 5,472,411 issued to H. Montag, et. al., on Dec. 5, 1995 teaches of a U-shaped flexible joint collar which wraps about the foot, heel and ankle.

As previously mentioned, while treatment of plantar fasciitis is one reason for needing immobilization of the ankle joint, there are other reasons, such as sprains, stress fractures and the like. These often require a walking boot. For example, the walking boot or splint commonly owned by Brown Medical of Brown, U.S. Pat. No. 6,361,514, but designed to be a universal splint suitable for either left or right foot usage. It is designed not only for usage to stretch the plantar fascia, but also for walking. As such it requires giving consideration to the patient's walking pattern, and a need to prevent slip and fall accidents. By walking pattern what is meant is the usual gait of people. Typically we walk with the foot landing on the heel and then rolling from the inside to the outside as the foot moves from heel to the ball joint to toes. This is called pronation.

Another common consideration for patient's wearing a ankle splint or boot to bed in order to stretch the plantar fascia is the frequent occurrence of a need to get up in the middle of the night, for example, to go to the bathroom. This may require walking down steps, and in so doing it is often difficult for a person to determine the edge of the step making it easy to miss the step entirely or to only have a slight portion of the heel catch on the step, causing frequent falls.

It can be seen there is, therefore, a need for a walking boot or splint which has an anti-skid attribute and which helps minimize the risk of slip and fall during use.

It is therefore a primary object of the present invention to provide an immobilizing boot or splint which has anti-skid characteristics that accommodate the normal walking pattern and which provide an extra gripping or anti-skid surface around the heel in order to prevent slip and falls while going up and down stairs.

It is a further object of the present invention to provide the above in walking splint using asymmetrical tread to improve grip and which can be adapted to a variety of different splint configurations.

As used here, the term “asymmetrical tread” refers to tread which has a lack of symmetry, that is, the tread on one side is slightly at variance with the tread on the other side so that it may not be identical if one were to make a fold line down the middle and fold one side upon the other. Put another way, if the folding did occur, there would be a variance because of the asymmetry. This variance enhances anti-skid properties during pronation and during traversing stairs.

### BRIEF SUMMARY OF THE INVENTION

A walking splint removably attachable to the lower leg and foot to substantially immobilize the ankle joint and provide stretching of the plantar fascia having the improved advantage that posterior shell of the walking splint has an asymmetrically positioned anti-skid tread on the exterior of the posterior shell, preferably under both the foot area and the heel area.

### BRIEF DESCRIPTION OF THE DRAWINGS

FIG. 1 is a perspective view of one example of the walking splint.

FIG. 2 is a bottom view of the walking splint showing the asymmetrical tread foot print.

FIG. 3 is a rear view of the splint of FIG. 1 showing the heel portion of the anti-skid tread.

FIG. 4 is a side view of the splint of FIG. 1 along line 4-4.

FIG. 5 is a side view of the splint of FIG. 1 along line 5-5.

### DETAILED DESCRIPTION OF THE PREFERRED EMBODIMENT

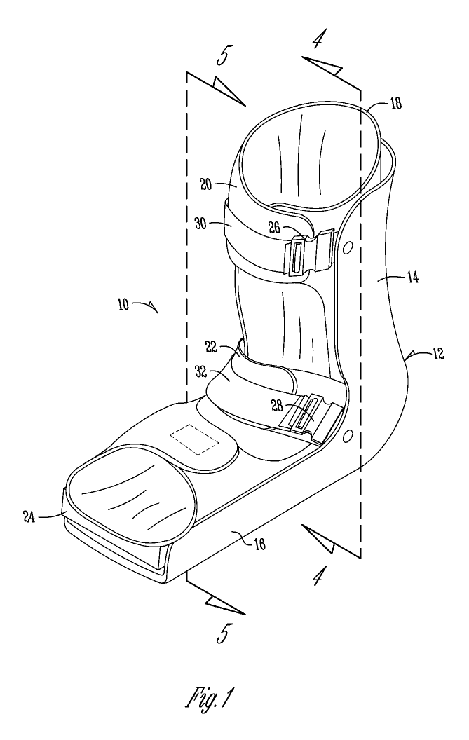

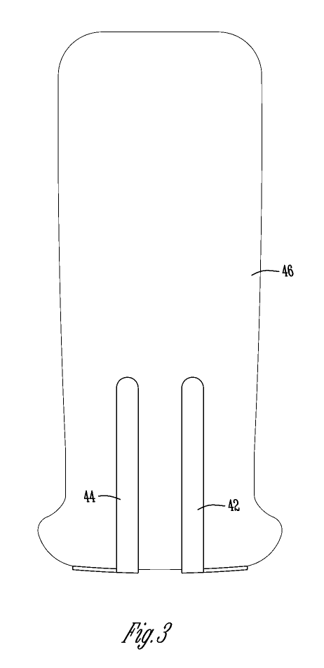

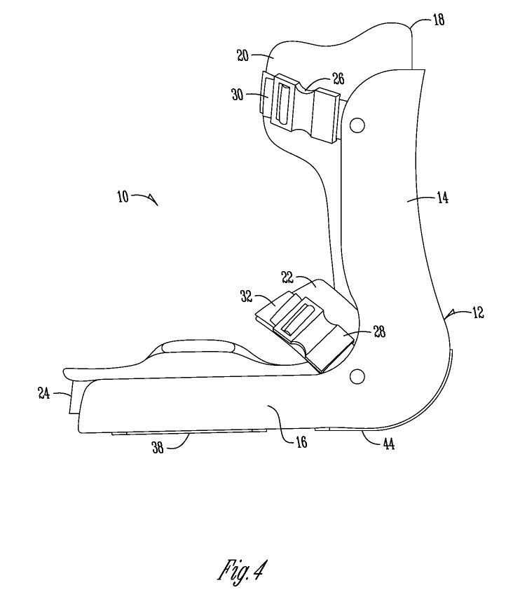

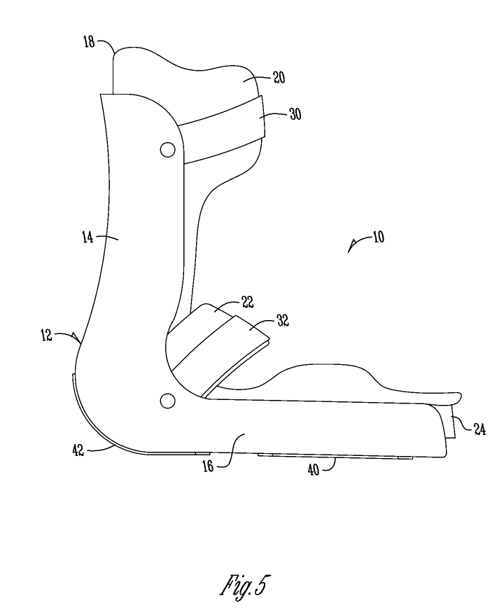

Shown in FIGS. 1-5 are a preferred form of the walking splint 10 of the present invention.

As seen best in FIGS. 1, 4 and 5, the splint 10 is comprised of a posterior rigid shell member 12 formed so that it is capable of encompassing the lower leg and foot of the patient. The posterior shell member 12 is the largest component of the splint. The posterior member 12 is generally an L-shaped shell that includes a vertical portion 14 that partially supports the posterior aspect of the leg from above the heel downwardly towards the heel and surrounds the heel to provide an underlying foot support area 16. The posterior member 14 has a heel recess formed therein to accommodate the heel of the wearer. Inserted into the shell formed by posterior shell 12 is a compressible foam wrap 18 which is comprised of a pad with a covering cloth that is useable with hook and pile fasteners such as those sold under the trademark Velcro™. Wrap 18 has strap portions 20 and 22 which may be used to variably adjust the compression around the foot or pad 18.

In order to assure that the ankle is bent at a proper 90° angle to stretch the Plantar Fascia, wedge pad 24 is inserted under the toe/ball joint area of the foot to keep the toes in an upward orientation. Buckles 26 and 28 pivotally attach to shell 14 so that straps 30 and 32 may tighten to secure the entire device 10 to the lower foot. We are aware that a previous patent of Brown Medical by two of the same inventors (others as well), U.S. Pat. No. 6,361,514, discloses at col. 3, line 32, a gripping sole to increase traction on the surface to minimize the possibility of slipping. But, what is missing from any of that disclosure is the importance of the gripping sole having asymmetrical tread thereon in order to maximize grip and minimize slip and fall.

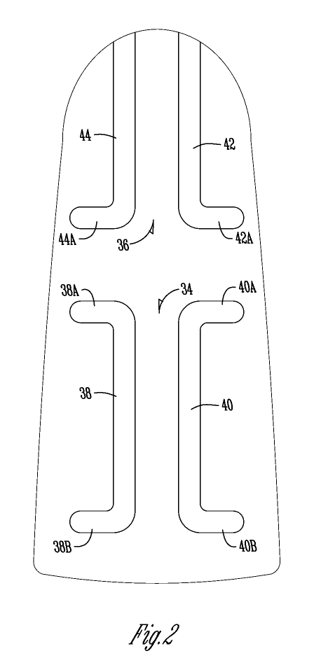

FIGS. 2, 3, 4 and 5 illustrate the asymmetrical anti-skid grip best. FIG. 2 shows the bottom side view of the posterior shell 14. In particular, it shows the footprint, of asymmetrical anti-skid tread. The tread has a foot portion 34 and a heel portion 36. As depicted in FIG. 2, the foot portion 34 comprises a pair of shaped treads 38 and 40 facing away from each other along the foot portion. They are generally of bracket configurations and are opposing each other so that there is a slight asymmetry, i.e. they converge slightly on one end and diverge slightly on the other end. The foot portion of the brackets diverge on one end and converge at the other to accommodate pronation since as the foot moves it generally moves from inside to outside during walking. Spaced apart from and behind the foot portion of asymmetrical tread 34 is the heel portion 36 of the asymmetrical tread. It likewise comprises two opposing faced configurations 42 and 44 which start under the heel, on the sole portion of the shell 14, and extend around the heel and slightly up the back 46 of the posterior shell 14. The portions of the tread terminate on their upper ends without any opposing facing short legs, since pronation is not an issue at that location. For convenience of description of the short legs of portion of the anti-skid foot portion are referred to at 38A and B and 40A and B and the short portion of the heel tread 36 is referred to as 44A and 42A.

In actual operation the device works in the following manner. The patient opens the buckles 26 and 28 and the straps 30 and 32, inserts their foot, adjusts foot and then closes pad straps 20 and 22 and woven straps 30 and 32, so the compression is at a comfortable but secure level. The patient may thereafter walk with the anti-skid asymmetrical treads, gripping to the ground or whatever surface is being walked upon. As the foot pronates, portions of the tread on the outside to provide a secure grip. If the patient is going up and down stairs and the heel only is on the stair portion, anti-skid grips 44, 42 catch and prevent slip and fall.

The anti-skid treads 34 and 36 may be adhered to the shell with any conventional glue adhesive such as a cyanoacrylate adhesive. They themselves may comprise any suitable polymeric material which functions as an anti-skid and can generally be described as a thermoplastic elastomer.

They may also have smooth surfaces or tread surfaces. The important point is they have the configuration of asymmetry and both a heel covering portion and a sole of the foot covering portion.

It can therefore be seen that the invention accomplishes at least all of its stated objectives.

1. In a walking splint that is removably attachable to the lower leg and foot to substantially immobilize the ankle joint while stretching the plantar fascia, comprising:
a posterior shell for the foot of substantially rigid material adapted to conform to the lower leg, the heel and the bottom of the foot; the improvement comprising:
an anti-skid asymmetric opposing treads positioned on the exterior of said posterior shell under the foot area located both under a heel portion of said posterior shell and a foot portion of said posterior shell, said anti-skid treads having an asymmetrical variance between the different portions of the tread one portion under the sole of the foot and the other portion of the tread under the heel of the foot.

2-4. (canceled)

5. The walking splint of claim 1 wherein the anti-skid tread has a footprint of two opposing position brackets facing away from each other located under the foot and two such members spaced apart from each other located under the heel.

6. The walking splint of claim 1 wherein at least two straps are attached to the posterior splint, vertically spaced from one another along the posterior shell and operable to adjustably circle the wearer's lower leg and foot to selectively apply tension to hold said members against the leg.

7. The walking splint of claim 1 wherein the support pad has tension adjustable straps.

8. The walking splint of claim 1 which includes a sole insert pad.
